(19) United States
(12) Patent Application Publication
(10) Pub. No.: US 2022/0378442 A1
(43) Pub. Date: Dec. 1, 2022

(54) DEVICES AND METHODS FOR ORIENTING A SURGICAL DEVICE

(71) Applicants: Johnson Yiu-Nam Lau, Houston, TX (US); Avalon Biomedical (Management) Limited, Hong Kong (CN)

(72) Inventors: Christian Xinshuo Fang, Hong Kong (CN); Kenneth Man-Chee Cheung, Hong Kong (CN); Kenny Yat Hong Kwan, Hong Kong (CN); Gar Hay Felix Sean Lau, Hong Kong (CN)

(21) Appl. No.: 17/775,532

(22) PCT Filed: Nov. 8, 2019
(86) PCT No.: PCT/US2019/060600

§ 371 Date: May 9, 2022

(51) Int. Cl. A61B 17/17; A61B 17/90

(52) U.S. Cl. CPC A61B 17/1742; A61B 17/90; A61B 90/11

(57) ABSTRACT

Devices and methods are provided that simplify and improve placement of stabilizing orthopedic pins, wires, and/or screws in the pelvis in minimally invasive surgery. Provided is a surgical instrument guide containing an adjustable friction clamp for positioning and securing a cannula, and a probe with a blunt, nonpenetrating tip. The probe and a surgical instrument held by the friction clamp are oriented at right angles to one another. In use, the blunt probe is introduced through a small incision and advanced by blunt dissection until the blunt tip is positioned at a desired anatomical landmark on the surface of the pelvis. A cannula inserted through the friction clamp is advanced until the tip of the cannula is within 2 cm of the blunt tip of the probe; an orthopedic pin, wire, and/or screw can then be inserted through the cannula to provide stabilization. The blunt probe may be cannulated.

### FIELD OF THE INVENTION

The field of the invention is devices and methods for orienting a surgical device relative to a human pelvis, in particular where the surgical device is a cannula.

### BACKGROUND

The following description includes information that may be useful in understanding the present invention. It is not an admission that any of the information provided herein is prior art or relevant to the presently claimed invention, or that any publication specifically or implicitly referenced is prior art. All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

While minimally invasive surgery (e.g. keyhole) surgery has reduced discomfort and recovery times, the use of small (frequently 2 cm or less) incisions complicates proper visualization of the surgical field and make proper instrument positioning challenging. When such surgical interventions are performed within a body cavity visualization can be assisted by introducing an absorbable gas (such as CO₂) and introducing a camera and illumination into the opened space. Such approaches, however, are not practical in enclosed or relatively enclosed spaces.

Towards that end various approaches have been implemented to assist in proper positioning of surgical devices for minimally invasive surgery in enclosed or relatively enclosed body compartments (e.g. interior of the skull, within or near joint spaces, etc.). For example, U.S. Pat. No. 5,776,144 describes an instrument that affixes a rounded ball to the skull, and mounting a swiveling holder for a surgical instrument on the rounded ball. Similarly, Great Britain Patent No. 903308 describes a device for positioning surgical instruments in which a base that is affixed to the skull supports a lattice of rotating rods that aid in supporting and orienting the surgical instrument. Such approaches, however, require a considerable area of hard bone for secure mounting of the base portion of these devices. In addition, the mounting process itself can generate small bone fragments, which is not desirable in or near articulating bone surfaces.

In a related approach, U.S. Pat. No. 8,636,745 describes a device that aids in the positioning of orthopedic pins used to stabilize fractures. The device has two cylindrical portions, mounted at right angles to one another in such a way that they can rotate. The cylindrical portions have through-holes through which such orthopedic pins can be placed. Following placement of one orthopedic, one of the cylindrical portions of the device is positioned such that the inserted pin passes through a set of through-holes. Rotation positions a set of through-holes in the remaining cylindrical portion for proper positioning of a second orthopedic pin. In such an approach, however, the accuracy of the placement of the second pin is dependent on the unguided placement of the first pin.

U.S. Pat. No. 5,776,143 describes a device for orienting a surgical tool by providing a base with three legs and that carries a swivel mount. In use the three legs are placed on markers positioned on the surface of the patient's body, and the swivel mount supports and orients the surgical instrument. However, if such markers are positioned on an area of the body surface that is relatively mobile or compressible (e.g. the surface of the abdomen) the accuracy of instrument placement could be impacted.

Collette and Cassard “The Tape Locking Screw Technique (TLS): A news ACL reconstruction method using a short hamstring graft” Orthopaedics and Traumatology: Surgery and Research (2011) 97:555-559 describe the use of tibial and femoral targeting devices that provide a support for a cannula and a probe, which are attached to opposing ends of an adjustable arcuate handle. The probe is positioned against the bone surface and stabilizes the handle, which is rotated to orient a cannula used for insertion of a surgical pin. The probes of such devices, however, typically have sharp tips that partially insert into the surface of the bone. While this provides stability, this impedes the ability to move the probe should initial placement prove to be incorrect and can also generate bone fragments (which are undesirable in or near opposing joint surfaces).

Thus, there is still a need for a simple and effective device that permits highly accurate and reproducible placement of surgical devices utilized in minimally invasive orthopedic surgery, while eliminating or minimizing unnecessary damage to the bone surface.

### SUMMARY OF THE INVENTION

The inventive subject matter provides devices and methods that simplify and improve the accuracy of stabilizing pin, wire, and/or screw placement during minimally invasive orthopedic surgery.

One embodiment of the inventive concept is a surgical instrument guide for positioning a surgical instrument (e.g. a surgical cannula) relative to a human pelvis, which includes a handle having a first terminus and a second terminus, a blunt probe with a blunt tip positioned at the first terminus, and a friction clap positioned at the second terminus. The friction clamp is configured to hold the surgical instrument so that it can slide within the friction clamp (i.e. is slidably engaged) when it is appropriately adjusted, and the friction clamp is oriented to orient the surgical instrument perpendicular to the blunt probe on placement in the friction clamp. The blunt tip of the blunt probe can be rounded or essentially flat. In blunt probes with a rounded tip blunt probe has a circular cross section that diminishes with increasing distance from the first terminus, and the blunt tip has a radius of curvature of less than or equal to about 25% or from about 5% to about 25% of the maximum radius of the circular cross section. In some embodiments the blunt probe is cannulated, for example to permit passage and placement of a stabilizing wire or pin.

Another embodiment of the inventive concept is a surgical instrument assembly for use with a human pelvis, which includes a surgical cannula with an outlet and a surgical instrument guide. The surgical instrument guide includes a handle having a first terminus and a second terminus, a blunt probe with a blunt tip positioned at the first terminus, and a friction clap positioned at the second terminus. The friction clamp is configured to hold the surgical instrument so that it can slide within the friction clamp (i.e. is slidably engaged) when it is appropriately adjusted, and the friction clamp is oriented to orient the surgical instrument perpendicular to the blunt probe on placement in the friction clamp. The blunt tip of the blunt probe can be rounded or essentially flat. In blunt probes with a rounded tip blunt probe has a circular cross section that diminishes with increasing distance from the first terminus, and the blunt tip has a radius of curvature of less than or equal to about 25% or from about 5% to about 25% of the maximum radius of the circular cross section. In some embodiments the blunt probe is cannulated, for example to permit passage and placement of a stabilizing wire. The surgical cannula is engaged with the friction clamp, and the outlet of the surgical cannula is positioned within 2 cm of the blunt tip when the surgical cannula is fully inserted in the friction clamp. The surgical cannula can include an inlet and an internal passage connecting the inlet and the outlet, and the inlet, internal passage, and outlet are dimensioned to guide a surgical pin or screw.

Another embodiment of the inventive concept is a method of positioning a surgical instrument against a pelvis using a surgical instrument guide that includes a first terminus and a second terminus, a blunt probe positioned at the first terminus and having a blunt tip, and a friction clap positioned at the second terminus and configured to slidably engage the surgical instrument. The friction clamp is oriented to orient the surgical instrument perpendicular to the blunt probe on engaging with the friction clamp such that the blunt tip is in contact with a bony surface of the pelvis. This surgical instrument guide is positioned such that the blunt tip can be moved across the bony surface of the pelvis to identify an anatomical landmark. The position of the surgical instrument guide on identification of the anatomical landmark. A surgical instrument (such as a cannula) is slidably engaged with the friction clamp, such that the surgical instrument is oriented perpendicular to the blunt probe, and is advanced through the friction clamp until an outlet of the surgical instrument is at a distance equal to or less than about 2 cm from the blunt tip of the blunt probe. In some embodiments the surgical instrument is engaged with the friction clamp prior to moving the blunt tip across the bony surface of the pelvis. If the surgical instrument is a cannula, such a cannula can include an inlet, an outlet, and an interior passage connecting the inlet and the outlet, and the method can include a step of introducing a surgical pin or screw into the interior passage. In a preferred embodiment the blunt tip of the blunt probe does not penetrate the body surface of the pelvis during the procedure. The blunt tip of the blunt probe can be essentially flat. or it can be rounded. If rounded the blunt tip can have a radius of curvature of less than or equal to about 25% or from 5% to 25% of the maximum radius of the circular cross section. In some embodiments the blunt probe is cannulated, and the method includes a step of introducing a stabilizing pin or wire into an interior passage of the blunt probe.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 depicts and embodiment of tool of the inventive concept, along with a cannula that can be used with such a tool. In use the cannula (150) is inserted through the friction clamp (140) of the tool such that it is oriented at a right angle to the blunt probe (120) used for accurate anatomical positioning.

FIG. 2 provides an enlarged view of a terminal portion of the blunt probe (200) and a view of the blunt tip (210) of the probe. While a flat tip is shown, any suitable non-penetrating configuration can be used.

FIG. 3 shows placement of a surgical guide of the inventive concept with a cannula placed within the friction clamp, providing proper positioning and orientation a surgical screw into the pelvis.

### DETAILED DESCRIPTION

Devices and methods of the inventive concept provide a stabilization and guidance tool that aid in the insertion of stabilizing pins, wires, and/or cannulated screws into the bones of the pelvis for internal fixation, using minimally invasive techniques. Devices of the inventive concept include a handle carrying a blunt, non-penetrative probe. This blunt probe can be introduced through a small (<1 cm) incision in the skin and moved through tissue by blunt dissection. Such a blunt, non-penetrative probe can be advanced until pelvic bone is contacted, the blunt nature of the probe tip preventing damage to the cortical layer of the bone and/or delicate joint tissue. Once the blunt probe is in contact with a desired anatomical landmark on the pelvis a cannula or similar instrument can be introduced through friction fitting or similar device that aligns the major (long) axis of the cannula or surgical device at a right angle to the blunt probe. The cannula or similar device can then be advanced through tissue to reach a desired target area, which has been identified at least in part by placement of the blunt tip of the blunt probe at the desired anatomical landmark. A fixation device (e.g. wire, pin, cannulated screw, etc.) is then passed through the interior passage of the cannula or similar device and inserted into the bone at the proper site and orientation.

One should appreciate that devices and methods of the inventive concept ensure that guide pins for cannulated screws can be placed in pelvic bone accurately towards any position within the pelvis with minimal chance of perforating the cortical bone and damaging the hip joint articular surface.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

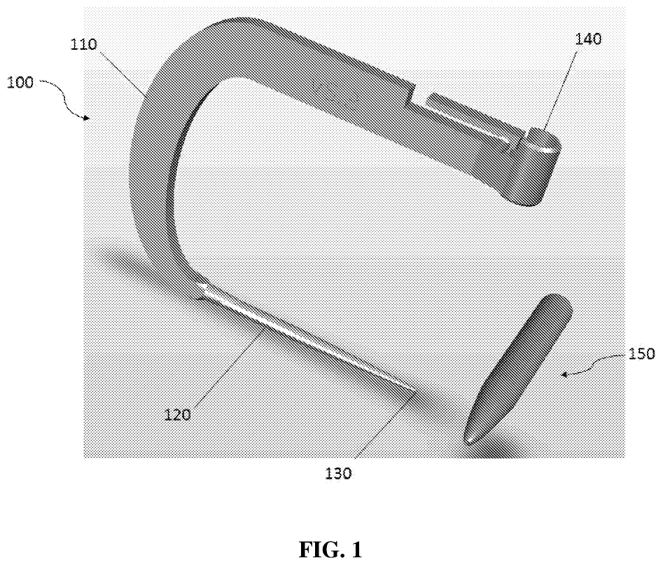

Devices, systems, and methods of the inventive concept provide or employ a novel targeting guide for minimal invasive, intuitive, reliable and safe placement of a surgical device (such as a cannula) that can be used for positioning and/or insertion of stabilizing pins, wires, and/or cannulated screws into the bony pelvis in one or more trajectories. An example of a targeting device or surgical guide of the inventive concept is shown in FIG. 1. The targeting device (100) includes of a blunt probe (120) that is coupled to one end of a handle (110) and that does not penetrate and/or affix to the bony surface of the pelvis when in use. Another end of the handle includes a friction clamp (140) or similar device that can engage a surgical cannula (150) (or similar device) such that the surgical cannula can progress by sliding through the friction clamp along its length (i.e. is slidably engaged), but can be secured by adjusting the tension of the clamp. The friction clamp is oriented such that the major axis of a surgical cannula that is engaged in the friction clamp is oriented at a right angle to the blunt (i.e. non-penetrating) probe. As shown in FIG. 1, in some embodiments the handle has a fixed length and configuration. In other embodiments the handle can be extendible so as to adjust the distance between the blunt probe (120) and the friction clamp (140) without changing the relative orientation between them.

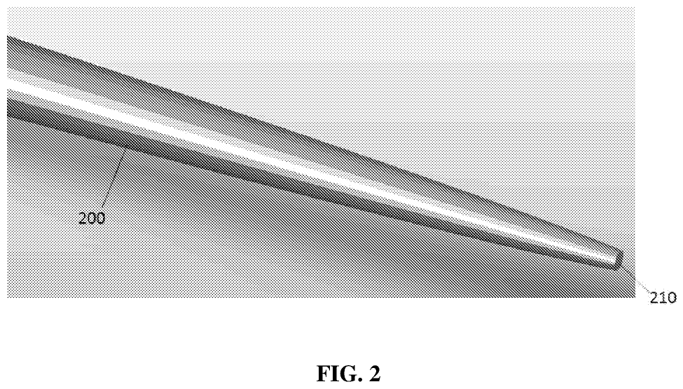

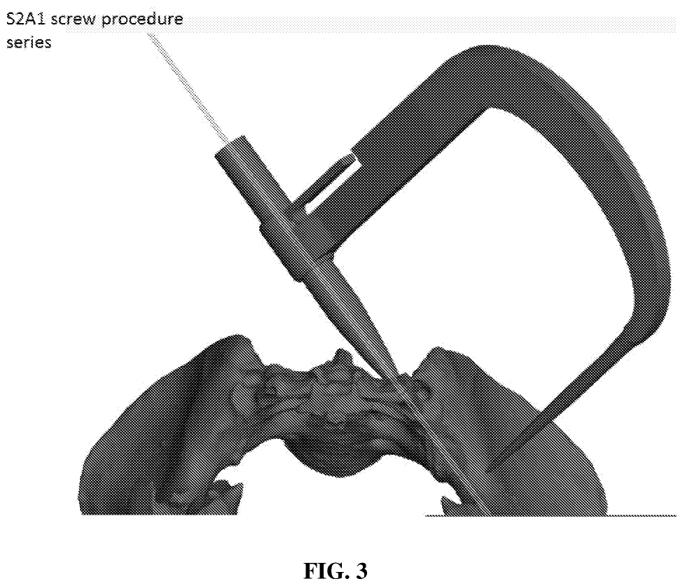

In use, the blunt probe is advanced through an incision in the skin into the pelvic region until the blunt (i.e. non-penetrating) tip (130) of the blunt probe contacts that bony surface of the pelvis, but does not penetrate into the bone. FIG. 2 provides an enlarged view of the blunt probe (200) and its blunt tip (210). In this example the blunt tip (210) is depicted as flat, but can have any suitable non-penetrating configuration. A user can then move the targeting device, translating the blunt probe over the surface of the pelvis until the desired anatomical landmark is identified (e.g. by tactile feedback through the probe, imaging, etc.). A surgical cannula can be engaged with the friction clamp during this positioning. Alternatively, the surgical cannula can engage with the friction clamp following this positioning. Upon placement of the blunt probe at the desired anatomical landmark on the surface of the pelvis, the surgical cannula is advanced through the friction clamp until the outlet of the cannula is in close proximity (e.g. at or within about 2 cm, or from about 0.5 cm to about 2 cm) to the blunt tip of the blunt probe). This positioning can be determined using imaging, by noting depth of penetration (e.g. through the use of indicia provided on the surgical cannula), or any suitable technique. FIG. 3 shows a typical placement of the surgical guide with a cannula placed within the friction clamp in an S2A1 screw placement procedure on a human pelvis.

Once the cannula is suitably positioned the friction clamp can be tightened in order to prevent further advancement of the cannula. In a preferred embodiment such tightening can be accomplished by releasing manual pressure applied to a portion of the friction clamp. Once the cannula is so secured a stabilizing pin, wire, and/or cannulated screw can be advanced through an inlet and internal channel of the cannula, exiting through an outlet of the cannula properly positioned for insertion into bony material of the pelvis.

In some embodiments the cannula can be withdrawn following insertion of the pin, wire, or screw into pelvis and the targeting guide or device reoriented for insertion of a second stabilizing pin, wire, and/or cannulated screw. In some embodiments reorientation is accomplished without moving the blunt tip of the blunt probe from the desired anatomical marker, for example by rotating the targeting guide while maintaining the position of the blunt probe against the bone. In other embodiments the blunt tip of the blunt probe can be moved to a different desired anatomical target prior to re-insertion of the cannula. In such embodiments the blunt probe can be removed through the initial incision and re-inserted through a new incision, or the blunt tip can be advanced to a new anatomical target while inserted by translation of the blunt tip of the blunt probe across the surface of the pelvis.

As noted above, the blunt probe has a blunt tip that is designed to not penetrate bone or damage the relatively thin cortex of the pelvic bone. Such a blunt tip can be essentially flat (e.g. deviating less than 10% from a plane oriented perpendicular to the major axis of the blunt probe). Alternatively, the blunt tip can be rounded. For example, the blunt probe can have a circular cross section that diminishes along the length of the probe as it progresses away from its point of coupling to the handle. The blunt tip of such a blunt probe can have radius (for a flat tip) or radius of curvature (for a rounded tip) that is less than about 25%, 20%, 15%, 10%, or 5% of the maximum radius of the blunt probe, or lie within a range between any of these values. The blunt probe can be designed to be inserted through a small (e.g. less than 1 cm) incision in the skin and advanced by blunt dissection through the deeper muscle tissues proximal to the pelvis, minimizing damage to both bone surface and/or muscle tissue. Such a blunt probe is configured to be placed in contact with one or more specific landmarks of the bony pelvis depending on the desired direction of the wire, pin, or cannulated screw.

As noted above, the handle has a friction or pressure clamp which permits slidable positioning of a surgical cannula inserted through an opening of the clamp. Such a cannula can be slid into a desired position, which a user can use to visualize the entry point of a guide pin when the cannula is locked into position. In a preferred embodiment the cannula can slide when finger pressure is applied to the friction clamp, and is fixed in position when such finger pressure is released.

When positioned at a desired orientation, a guide pin or screw of desired diameter can be drilled into the bone as it positioned via the cannula, oriented perpendicular to the blunt probe and at a distance of less than 2 cm, from 0.5 cm to 2 cm, or, preferably, from 10 to 15 mm from the blunt tip of the blunt probe.

In some embodiments the probe can also be cannulated (i.e. include an inlet, a terminal outlet, and an internal passage connecting the inlet with the outlet) to allow for placement of a stabilizing device (such as a K-wire) for temporary stabilization of the cannula insertion of a screw guidance guide pin.

As noted above, in some embodiments the position of blunt probe and/or the cannula can be determined by imaging. Towards that end all or a portion of the guidance device and/or the cannula can include a radio-opaque material (e.g. stainless steel). In some embodiments a device and/or system of the inventive concept can be composed entirely of radio-opaque material. In other embodiments a device and/or system of the inventive concept can be composed of radiolucent material (e.g. polymer) and incorporate radio-opaque portions for imaging purposes. Similarly, in some embodiments a blunt probe and/or cannula of devices and systems of the inventive concept can include visible indicia along their length that provide a user with information regarding the depth of insertion in order to facilitate proper positioning.

In practice there are multiple uses of the devices for screw placement in the pelvis, including positioning and insertion of an S2-Alar-iliac screw (posterior entry), a posterior column screw (antegrade pelvic brim insertion), an anterior column screw (antegrade ileum outer table insertion), an iliac column screw (retrograde AIIS insertion), an iliac column screw (antegrade PIIS insertion), and/or a posterior column screw (retrograde ischial tuberosity insertion).

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

1. A surgical instrument guide for positioning a surgical instrument relative to a human pelvis, comprising:
a handle having a first terminus and a second terminus;
a blunt probe positioned at the first terminus and comprising a blunt tip; and
a friction clap positioned at the second terminus and configured to slidably engage the surgical instrument, wherein the friction clamp is oriented to orient the surgical instrument perpendicular to the blunt probe upon engagement with the friction clamp.

2. The surgical instrument guide of claim 1, wherein the friction clamp is dimensioned to slidably engage a surgical cannula.

3. The surgical instrument guide of claim 1, wherein the blunt tip is essentially flat.

4. The surgical instrument guide of claim 1, wherein the blunt tip is rounded.

5. The surgical instrument guide of claim 4, wherein the blunt probe has a circular cross section that diminishes with increasing distance from the first terminus, and wherein the blunt tip has a radius of curvature of less than or equal to about 25% of the maximum radius of the circular cross section.

6. The surgical instrument guide of claim 5, wherein the blunt tip has a radius of curvature of from about 5% to about 25% of the maximum radius of the circular cross section.

7. The surgical instrument guide of claim 1, wherein the blunt probe is cannulated.

8. A surgical instrument assembly for use with a human pelvis, comprising:
a surgical cannula comprising an outlet; and
a surgical instrument guide comprising:
a handle having a first terminus and a second terminus;
a blunt probe positioned at the first terminus and comprising a blunt tip; and
a friction clap positioned at the second terminus and configured to slidably engage the surgical cannula, wherein the friction clamp is oriented to orient the surgical cannula perpendicular to the blunt probe upon engagement with the friction clamp,
wherein the outlet of the surgical cannula is positioned within 2 cm of the blunt tip when the surgical cannula is fully inserted in the friction clamp.

9. The surgical instrument assembly of claim 8, wherein the surgical cannula comprises an inlet and an internal passage connecting the inlet and the outlet, and wherein the inlet, internal passage, and outlet are dimensioned to guide a surgical pin or screw.

10. The surgical instrument assembly of claim 8, wherein the blunt tip is essentially flat.

11. The surgical instrument assembly of claim 8, wherein the blunt tip is rounded.

12. The surgical instrument assembly of claim 11, wherein the blunt probe has a circular cross section that diminishes with increasing distance from the first terminus, and wherein the blunt tip has a radius of curvature of less than or equal to about 25% of the maximum radius of the circular cross section.

13. The surgical instrument assembly of claim 11, wherein the blunt tip has a radius of curvature of from about 5% to about 25% of the maximum radius of the circular cross section.

14. The surgical instrument assembly of claim 8, wherein the blunt probe is cannulated.

15. A method of positioning a surgical instrument against a pelvis, comprising:
positioning a surgical instrument guide comprising a handle having a first terminus and a second terminus, a blunt probe positioned at the first terminus and comprising a blunt tip, and a friction clap positioned at the second terminus and configured to slidably engage the surgical instrument,
wherein the friction clamp is oriented to orient the surgical instrument perpendicular to the blunt probe upon engagement with the friction clamp such that the blunt tip is in contact with a bony surface of the pelvis;
moving the blunt tip across the bony surface of the pelvis to identify an anatomical landmark;
maintaining position of the surgical instrument guide upon identification of the anatomical landmark;
slidably engaging the surgical instrument with the friction clamp, such that the surgical instrument is oriented perpendicular to the blunt probe; and
advancing the surgical instrument through the friction clamp until an outlet of the surgical instrument is at a distance equal to or less than about 2 cm from the blunt tip.

16. The method of claim 15, wherein the surgical instrument is engaged with the friction clamp prior to moving the blunt tip across the bony surface of the pelvis.

17. The method of claim 15, wherein the surgical instrument is a surgical cannula.

18. The method of claim 17, wherein the surgical cannula comprises an inlet and an interior passage connecting the inlet and the outlet, and further comprising the step of introducing a surgical pin or screw into the interior passage.

19. The method of claim 15, wherein the blunt tip does not penetrate the body surface of the pelvis.

20. The method of claim 15, wherein the blunt tip is essentially flat.

21. The method of claim 15, wherein the blunt tip is rounded.

22. The method of claim 21, wherein the blunt probe has a circular cross section that diminishes with increasing distance from the first terminus, and wherein the blunt tip has a radius of curvature of less than or equal to about 25% of the maximum radius of the circular cross section.

23. The method of claim 22, wherein the blunt tip has a radius of curvature of from about 5% to about 25% of the maximum radius of the circular cross section.

24. The method of claim 15, wherein the blunt probe is cannulated, and comprising a step of introducing a stabilizing pin or wire into an interior passage of the blunt probe.
